(19) United States
(12) Patent Application Publication
(10) Pub. No.: US 2011/0257691 A1
(43) Pub. Date: Oct. 20, 2011

(54) LOCKING CANNULATED BONE SCREW SYSTEM

(75) Inventors: Chet Sutterlin, Long Boat Key, FL (US); Joseph Gleason, Eagan, MN (US); Dan McPhillips, Ham Lake, MN (US); Tim Walnofer, Stillwater, MN (US)

(73) Assignee: SPINEOLOGY, INC., St. Paul, MN (US)

(21) Appl. No.: 12/951,450

(22) Filed: Nov. 22, 2010

Related U.S. Application Data
(60) Provisional application No. 61/263,222, filed on Nov. 20, 2009.

(51) Int. Cl. A61B 17/86

(52) U.S. Cl. 606/305

(57) ABSTRACT

A cannulated bone screw having a cannula or lumen defined herein is disclosed. The screw may be fully or partially threaded and may have a cancellous thread form. Alternatively, the head of the screw may be generally flat on its undersurface. The screw head may further include an anti-backout feature to ensure good contact with the mating bone surface. The screw may further include an anti-rotation feature on the underside of the screw head. It is further disclosed that insertion and seating of the screw may be accomplished via locking engagement between jaws on the screw driver and a short raised feature on the head of the screw. The screws may be fully or partially threaded. A rescue screw is additionally disclosed.

### PRIORITY

This application claims the benefit of U.S. Provisional Application Ser. No. 61/263,222, filed on Nov. 20, 2009, which is hereby incorporated by reference in its entirety herein.

### FIELD

The present invention relates generally to a bone screw for bony fixation. More particularly the present invention relates to a locking cannulated bone screw system and its method of use.

### BACKGROUND

Bone screws are used to stabilize and secure bones. One particular type of bone screw is a facet screw. Facet screws are used to stabilize the spine by assisting spinal fusion by transfacet fixation. Many facet screw systems currently exist. One commercially available facet screw system is the CHAMELEON Fixation System by SpineFrontier, Inc. TranS1 Facet Screws are another example of commercially available facet screws. However, conventional facet screw systems have several disadvantages and as such there is a need for an improved facet screw.

One such disadvantage is premature screw fall out from the screw driver. As the surgeon begins to implant the screw, most conventional screws are subject to disengaging from the driver making it difficult for the surgeon to easily and accurately implant the screw.

Secondly, conventional screws are subject to backing out of the bone, or becoming dislodged from the bone once seated. A dislodged screw can be dangerous to the anatomy surrounding the vertebra, including the spinal cord. At the very least, a dislodged screw is ineffective in its intended purpose of stabilizing the spine to promote and assist in fusion.

Thirdly, most conventional screws include a bulbous seating surface. The bulbous surface results in the seating torque being proportional to the depth the screw is driven into the bone resulting in poor tactile feedback for the surgeon. Poor tactile feedback makes it difficult for the surgeon to know when the screw has been seated and to what depth.

It is desirable to overcome these disadvantages of the currently available screw systems.

### SUMMARY

The bone screw of the present invention may be a cannulated screw having a cannula or lumen defined herein. In one embodiment of the present invention, the screw may be fully or partially threaded and may have a cancellous thread form. In another embodiment of the present invention, the head of the screw may be generally flat on its undersurface.

According to one aspect of the present invention, the screw head may further include an anti-backout feature to ensure good contact with the mating bone surface. According to an embodiment of the present invention, screws may include an anti-rotation feature on the underside of the screw head.

According to another embodiment of the present invention, insertion and seating of the screw may be accomplished via locking engagement between jaws on the screw driver and a short raised feature on the head of the screw. This feature ensures secure purchase on the screw such that the physician can more readily control it as needed during the insertion procedure.

In one embodiment of the present invention, the screws of the present invention may be machined from a titanium alloy. In one embodiment a bone screw of the present invention may have a major diameter in the range of about 5.25 mm and a minor diameter in the range of about 3.3 mm.

According to an embodiment of the present invention, the screw length may be in the range of about 25 to 35 mm. In one embodiment screw lengths may be available in the range of about 5 mm increments.

In one aspect of the present invention, the screws may be partially threaded. In a partially threaded embodiment the screws may have an unthreaded lag length in the range of about 10 mm.

In yet another embodiment, the bone screw system of the present invention may also include a rescue screw for the unlikely event wherein screw threads in the bone become stripped during implantation. In one embodiment, the rescue screw may have a major diameter in the range of about 6.25 mm and screw lengths ranging from about 25 to 35 mm in about 5 mm increments.

In one embodiment of the present invention, the cannulation diameter of the screw may be in the range of about 1.2 mm and the head diameter in the range of about 8 mm. Other lengths and diameters are contemplated and are within the scope of this invention.

The above summary is not intended to limit the scope of the invention, or describe each embodiment, aspect, implementation, feature or advantage of the invention. The detailed technology and preferred embodiments for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a side perspective view of a threaded screw according to one embodiment of the present invention.

FIG. 2 depicts a top perspective view of a screw head according to an embodiment of the present invention.

FIG. 3 depicts an embodiment of a driver and screw according to an embodiment of the present invention.

FIG. 4 depicts an embodiment of a screw captured by a driver according to an embodiment of the present invention.

FIG. 5 depicts an embodiment of a screw captured and fully retracted in a driver according to an embodiment of the present invention

FIG. 6 depicts a side perspective view of a lag screw according to an embodiment of the present invention.

### DETAILED DESCRIPTION

In the following descriptions, the present invention will be explained with reference to various example embodiments; nevertheless, these example embodiments are not intended to limit the present invention to any specific example, embodiment, environment, application, or particular implementation described herein. Therefore, descriptions of these example embodiments are only provided for purpose of illustration rather than to limit the present invention. The invention is to cover all modifications, equivalents, and alternatives falling within the scope of the invention as defined by the appended claims

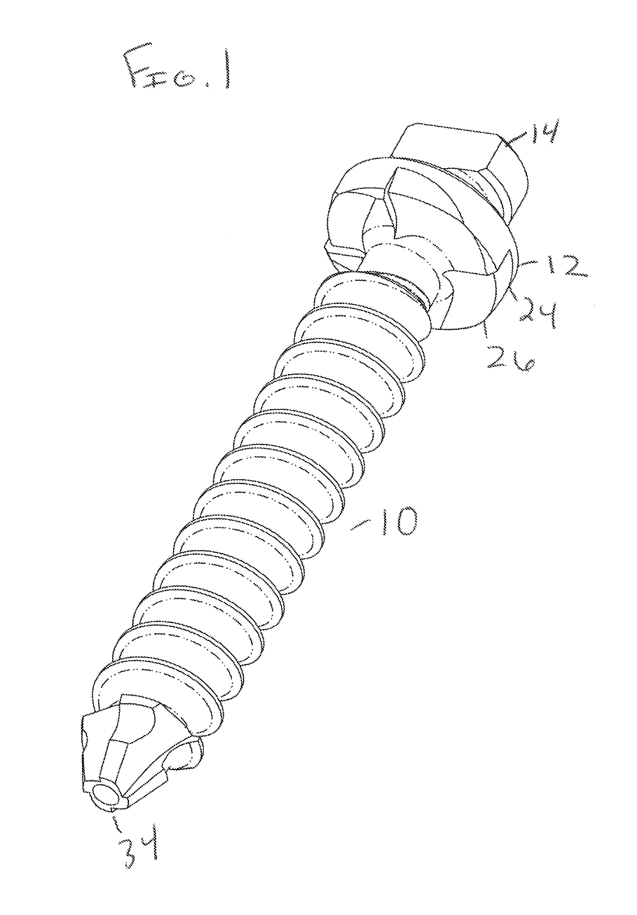

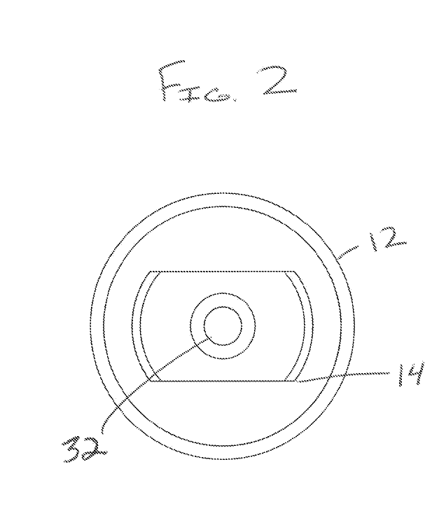

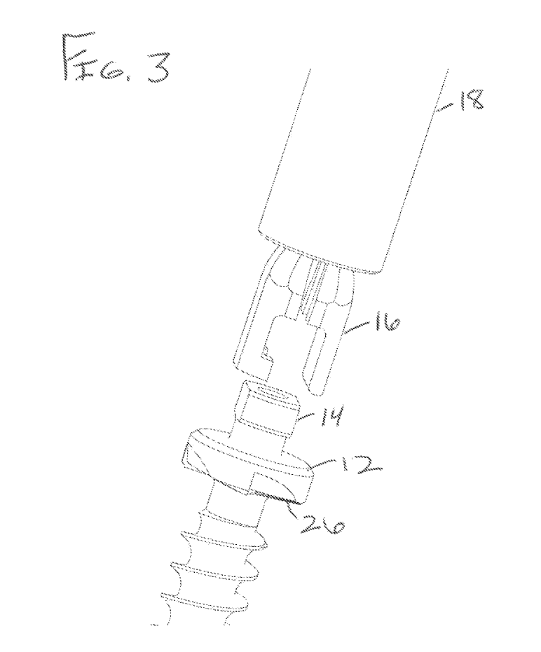

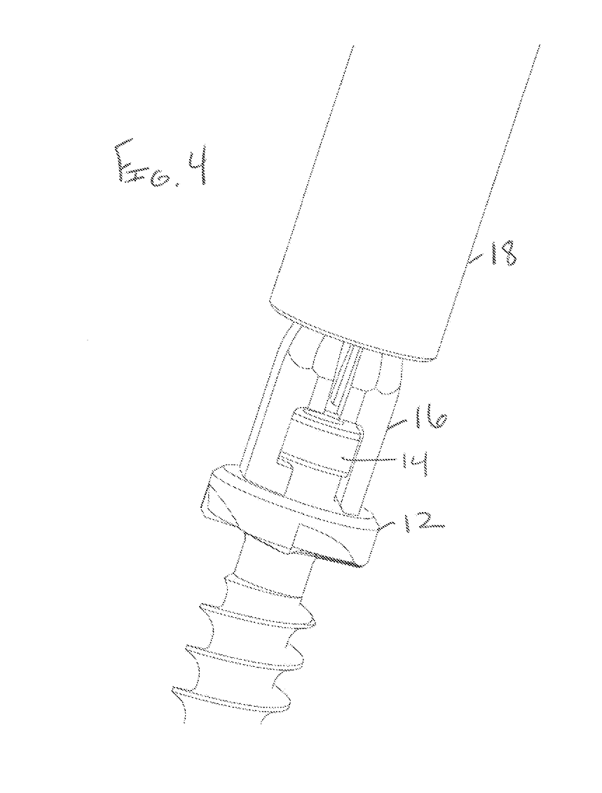

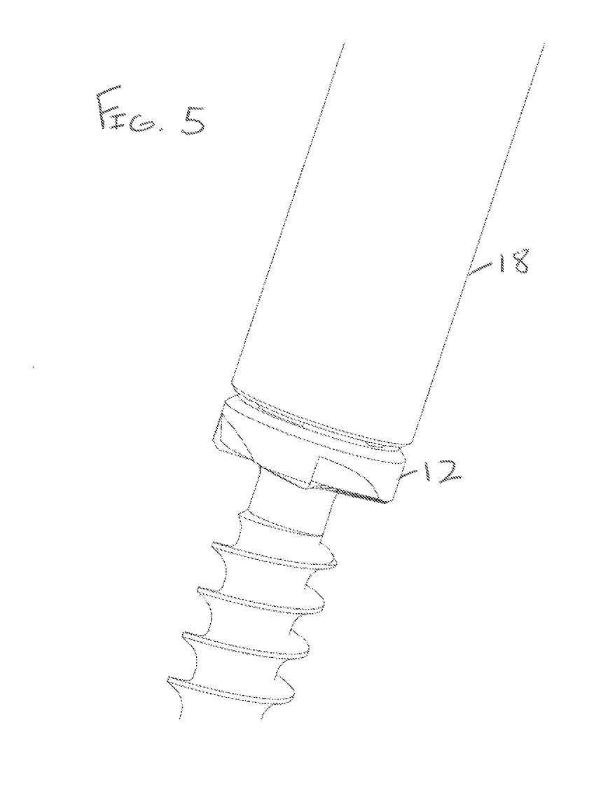

As can be seen in FIG. 1, bone screw 10 of the present invention may include screw head 12. In an embodiment of the present invention, head 12 may further include a driver engagement portion 14 as shown in FIG. 2. Driver engagement 14 may engage capture portion 16, such as for example an undercut feature, of the screw driver 18, as shown in FIG. 3. In an embodiment, capture portion 16 may be configured as a collar that surrounds the periphery of driver engagement 14. Once engaged, as can be seen in FIGS. 4 and 5, screw 10 may be captured by driver 18 until manually released by the user, such as for example by deploying shaft 20 from housing 22. Driver engagement 14 prevents loss of connectivity of screw 12 to screw driver 18 from accidental retraction of driver 18.

Screw head 12 may be configured in a generally flat seating geometry 24. Flat geometry 24 provides a stop against bone. In contrast, most facet screws are configured with a bulbous seating geometry. Such bulbous shapes result in a screw seating torque that is proportional to how far into the bone the screw is seated. Flat seating geometry 24 provides higher torque at seating which provides greater tactile feedback to the surgeon than does the bulbous seating geometry. Flat seating geometry 24 results in a relatively sudden increase in insertion torque which in turn provides a tactile indicator of screw 10 seating. Head 12 also may include an increased head diameter over conventional screws. The head diameter may be in the range of about 7 to 9 mm. This increased head diameter prevents the screw head from being pulled through the cortical bone after contact and provides the surgeon with tactile feedback that the screw has made contact with bone and is implanted to the desired depth.

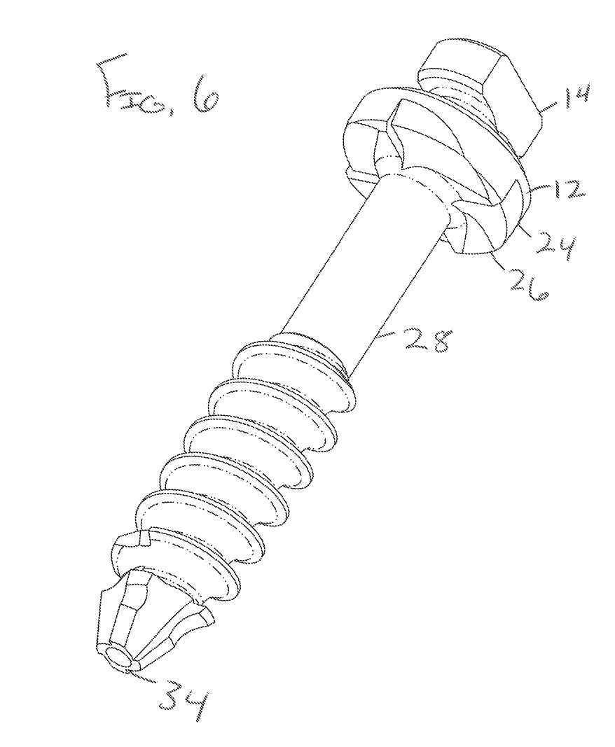

Screw 10 may also include an anti-backout portion 26 on the underside of head 12. In an embodiment, anti-backout portion 26 may include a directional sloping feature much like a propeller that only allows one directional motion. According to this embodiment, screw 10 may be driven into bone in a clockwise direction. As screw 10 is driven into the bone, anti-backout feature 26 may abrade and deflect bone. If screw 10 is turned in a counterclockwise motion, anti-backout feature 26 may grab and dig into the bone, thus preventing screw 10 from backing out once implanted. Once screw 10 is seated in a clockwise direction, attempting to unscrew screw 10 in a counter-clockwise direction causes anti-backout feature 26 to dig into the cortical bone and lock screw 10 into place. FIG. 6 depicts another embodiment of screw 10 wherein screw 10 includes lag 28. The length of lag 28 may be in the range of about 10 mm.

According to one aspect of the present invention as is shown in FIGS. 4 and 5, driver 18 may distribute force to flat head 12 resulting in direct pressure to anti-backout feature 26. Such direct pressure may assist in the seating of screw 10. In one embodiment, driver 18 is manually manipulated by a hand powered tool. In another embodiment, driver 18 is power manipulated by a powered drill driver tool.

Screw 10 is not limited to use in the spine and may be used in other bony locations as desired by the physician user. In one embodiment, the bone screw system of the present invention may be used to stabilize the spine to assist in spinal fusion. According to such use, the screw system of the present invention may be placed either before or after the fusion procedure, depending on physician preference. Cannulated bone screws generally require the drilling of a pilot hole. A guide wire may be placed into the desired position and then a pilot hole may be drilled over the guide wire. Cannulated screw 10 with a cannula or lumen running between proximal end 32 and distal end 34 may then be placed over the guide wire and screwed into the pilot hole. According to one embodiment of the present invention the fusion procedure may include the following steps in the order desired by the physician and may or may not include all of the detailed steps:

First, a midline point may be identified that provides access to the most inferior aspect of the desired facet joints. Then, an incision, in the range of about less than 5 mm, may be made at the identified midline point. Next, a guide pin may be passed through the incision and advanced to the inferior aspect of the facet joint to approximately the point where the joint contacts the next most inferior lamina. Then, the pin may be tapped into the joint to anchor the pin.

Next, a dilator and cannula may be advanced over the guide pin to the facet. The cannula may be seated and then the dilator may be removed. Next a cannulated cutting tool may be slid, such as for example a rasp, burr, curette, etc, over the pin to the facet joint. In an embodiment, the cannulated cutting tool may include a fluted tip such that the cutting tool cuts to the front and sides of the cutting tip. Using the cannulated cutting tool, the most inferior aspect of the inferior facet, the joint surface and the lamina may be decorticated.

Then, bone graft or bone graft substitute, such as for example, BMP, may be placed in the cannula and tamped into the desired location at the decorticated site. The instruments may then be removed and the procedure may be repeated on the contralateral side.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A bone screw for use in bone and tissue in a mammal comprising:
a bone screw head having a proximal driver engaging portion, the driver engaging portion configured to operably engage a driver such that the bone screw is secured to the driver until manually released.

2. The bone screw of claim 1 wherein the driver is operably engaged around the periphery of the driver engaging portion.

3. The bone screw of claim 1 wherein the bone screw head is configured in a generally flat geometry.

4. The bone screw of claim 1 wherein the bone screw head includes a one directional anti-backout portion configured to abrade bone in one direction and engage bone in the opposite direction.

5. A bone screw for use in bone and tissue in a mammal comprising:
a bone screw head having a one directional anti-backout portion configured to abrade bone in one direction and engage bone in the opposite direction.

6. The bone screw of claim 5 wherein the anti-backout portion is configured as a directional sloping propeller.

7. The bone screw of claim 5 wherein the bone screw head is configured in a generally flat geometry.

8. The bone screw of claim 5 wherein the bone screw head includes a proximal driver engaging portion, the driver engaging portion configured to operably engage a driver such that the bone screw is secured to the driver until manually released.
